(19) United States
(12) Patent Application Publication
(10) Pub. No.: US 2022/0282605 A1
(43) Pub. Date: Sep. 8, 2022

(54) ACCELERATED PROGRAMED SOURCE ROCK PYROLYSIS

(71) Applicant: SAUDI ARABIAN OIL COMPANY, Dhahran (SA)

(72) Inventors: Bandar Ismail Ghassal, Dhahran (SA); Salman Mohammed Qathami, Dhahran (SA); Khalid Raddad Malki, Dhahran (SA)

(73) Assignee: SAUDI ARABIAN OIL COMPANY, Dhahran (SA)

(21) Appl. No.: 17/192,344

(22) Filed: Mar. 4, 2021

(51) Int. Cl. E21B 43/241; E21B 49/08; G01N 33/28

(52) U.S. Cl. CPC E21B 43/241; E21B 49/088; G01N 33/2823

(57) ABSTRACT

A method includes placing a rock sample in an inert environment in an oven, heating the rock sample in the oven at about 300° C. for between about 3 and 4 minutes, increasing the temperature to about 750° C. at a rate ranging between 50° C. per minute and 60° C. per minute, and detecting pyrolysis material from the rock sample by a flame ionization detector.

### BACKGROUND

Rock analysis is typically performed to determine characteristics of a formation. Formation characteristics may be used for planning a drilling phase of the formation and/or for planning a production phase of a reservoir in the formation. Rock analysis may include, for example, lab testing on samples of formation rock and logging operations, where testing equipment is sent downhole to retrieve rock characteristic data from downhole locations.

One technique for rock evaluation is pyrolysis, which may be used to evaluate the type and maturity of organic matter in a rock sample and oil/gas generation potential from the rock. In this technique, an organically rich source rock may be heated in a pyrolysis oven using a heating schedule that may include multiple heating phases. As the rock sample is heated, different hydrocarbon components within the sample may react under the heated conditions, which may be detected and plotted in a pyrogram (a graph of yield v. heating time). For example, free hydrocarbons in a rock sample may be volatized under a first heating phase and detected. Detected solvent extractable hydrocarbons (e.g., bitumen) may be measured as an S₁ peak on the pyrogram from the pyrolysis process. In a second heating phase (at temperatures greater than the first heating phase) of the pyrolysis process, heavier and/or non-solvent extractable hydrocarbons (e.g., kerogen) may be volatized or cracked, which may be measured as an S₂ peak on the pyrogram. The temperature at which the S₂ peak reaches its maximum is referred to as Tₘₐₓ. Further, during conventional pyrolysis, CO₂ issued from cracked organic material may be trapped and then detected by an infrared detector. The detected CO₂ may be measured as an S₃ peak on the pyrogram.

Different parameters may be derived from pyrolysis analysis, including, for example, the total organic carbon (TOC), the hydrogen index (HI), the oxygen index (OI), and the production index (PI) of the rock sample. The HI may be calculated from S₂/TOC (mgHC/gTOC) and may be used to characterize the origin of the organic matter in the rock sample. The OI may be calculated from S₃/TOC (mgCO₂/gTOC) and correlates with the ratio of oxygen to carbon in the rock sample. The PI may be calculated from the equation S₁/(S₁+S₂) (no unit) and may be used to characterize the evolution level of the organic matter in the rock sample.

### SUMMARY OF INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments of the present disclosure relate to pyrolysis methods that include accelerated heating schedules. For example, some methods disclosed herein include placing a rock sample in an inert environment in an oven, heating the rock sample in the oven at about 300° C. for between about 3 and 4 minutes, increasing the temperature to about 750° C. at a rate ranging between 50° C. per minute and 60° C. per minute, and detecting pyrolysis material from the rock sample by a flame ionization detector.

In another aspect, embodiments of the present disclosure relate to methods that include subjecting a rock sample to a pyrolysis process lasting less than 15 minutes. For example, pyrolysis processes disclosed herein may include a first heating phase of heating the rock sample at an initial temperature of about 300° C., a second heating phase of heating the rock sample from the initial temperature to a final temperature ranging between 650° C. and 750° C. at a rate of 50° C. per minute, and detecting pyrolysis material from the rock sample during the first and second heating phases. A pyrogram may be generated from the pyrolysis process that includes a plot of an amount of the detected pyrolysis material as a function of time.

In yet another aspect, embodiments of the present disclosure relate to systems that include a pyrolysis oven, a flame ionization detector, and a computing system in communication with the pyrolysis oven and the flame ionization detector. The flame ionization detector may include a burner fluidly connected to a heating chamber in the pyrolysis oven and a fuel gas and a collector electrode positioned above the burner. The computing system may have computer readable instructions for controlling a temperature in the pyrolysis oven according to a heating schedule that includes heating the pyrolysis oven at an initial temperature for between 3 and 4 minutes in a first heating phase, and in a second heating phase, increasing the temperature in the pyrolysis oven at a rate ranging between 50° C. per minute and 60° C. per minute to a final temperature. The computing system may further include instructions for generating a plot of an amount of detected pyrolysis material as a function of time based on changes in measured electrical flow through the collector electrode.

Other aspects and advantages of the invention will be apparent from the following description and the appended claims.

### BRIEF DESCRIPTION OF DRAWINGS

FIG. 1. shows a schematic diagram of an example pyrolysis system according to embodiments of the present disclosure.

FIG. 2 shows an example of a pyrolysis method according to embodiments of the present disclosure.

FIG. 3 shows an example of a pyrogram generated from a pyrolysis method according to embodiments of the present disclosure.

FIG. 4 shows a pyrogram comparing results generated from a conventional pyrolysis method and a pyrolysis method according to embodiments of the present disclosure.

FIG. 5 shows a comparison of Tₘₐₓ values determined from a conventional pyrolysis method and a pyrolysis method according to embodiments of the present disclosure.

FIG. 6 shows a comparison of S₂ values determined from a conventional pyrolysis method and a pyrolysis method according to embodiments of the present disclosure.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

Methods disclosed herein include pyrolysis temperature programs that may be used to determine pyrolysis parameters faster than conventionally used methods and more accurate Tₘₐₓ values. During disclosed pyrolysis methods, organic matter within a rock sample may be decomposed by heating the rock sample in an inert atmosphere (e.g., in the absence of oxygen) and detected using one or more detectors. The detected pyrolysis material may be used to determine the richness and maturity of hydrocarbons within the rock sample (often described in terms of pyrolysis parameters such as S₁ peak, S₂ peak, TOC (total organic carbon), HI (hydrogen index), PI (production index), Tₘₐₓ, etc.). Pyrolysis material analysis may be used, for example, to determine potential of the rock sample and/or formation from which the rock sample came as hydrocarbon source rock. By using pyrolysis methods according to embodiments of the present disclosure, pyrolysis analysis may be performed faster and more accurately compared to conventional methods, which may improve the speed and accuracy of overall source rock assessments.

In accordance with embodiments disclosed herein, pyrolysis methods may be performed by heating a rock sample in a pyrolysis oven according to an accelerated heating schedule and using one or more detectors to detect pyrolysis material as the rock sample is heated. Pyrolysis ovens may include conventionally used pyrolysis ovens or other ovens having a heating chamber and a system for providing the heating chamber with an inert environment (e.g., a fluidly connected gas source such as helium or H₂ and/or a vacuum). Further, one or more detectors may be fluidly connected to the heating chamber such that an effluent from the pyrolysis decomposition (pyrolysis material) may flow from the heating chamber to the one or more detectors to be detected and analyzed.

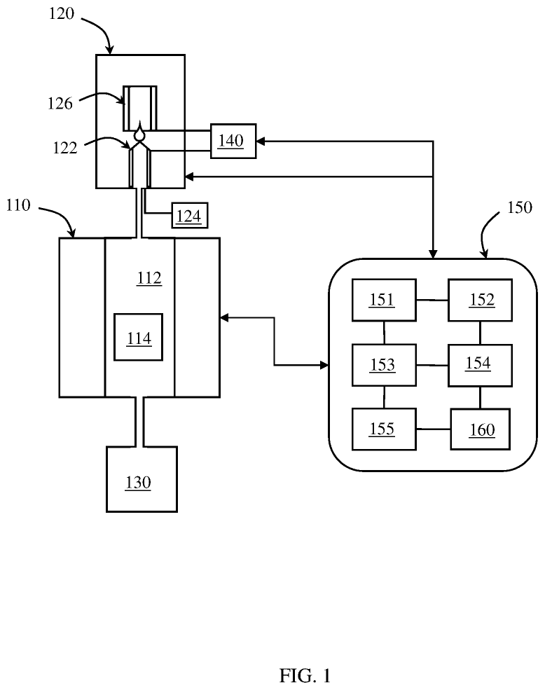

For example, FIG. 1 shows a schematic diagram of a system 100 that may be used to perform methods in accordance with one or more embodiments of the present disclosure. In one or more embodiments, one or more of the modules and/or elements shown in FIG. 1 may be omitted, repeated, and/or substituted. As shown in FIG. 1, the system 100 may include a pyrolysis oven 110 and a connected flame ionization detector (FID) 120. The pyrolysis oven 110 may include a heating chamber 112, in which a rock sample 114 may be placed. An inert gas source 130 may be fluidly connected to the heating chamber 112 to provide an inert environment in the heating chamber 112. For example, hydrogen gas or other inert gas may be pumped from the inert gas source 130 to the heating chamber 112 to fill the heating chamber 112 during pyrolysis. In some embodiments, a vacuum may be provided to remove oxygen from the heating chamber 112 prior to or during pumping the inert gas into the heating chamber 112.

In the embodiment shown, a single FID 120 is shown fluidly connected to the heating chamber 112 to provide a single detector connected to the pyrolysis over 110. However, in one or more embodiments, additional and/or other types of detectors may be fluidly connected to the heating chamber 112 to detect pyrolysis material generated during pyrolysis of the rock sample 114. For example, in some embodiments, an infrared (IR) detector may be connected to the pyrolysis oven 110 in addition to the FID 120. The FID 120 may include a burner 122 fluidly connected to the heating chamber 112 and a fuel gas source 124, where the fuel gas source 124 may provide a fuel gas to the burner to keep the flame lit, and where a fluid connection between the heating chamber 112 and burner 122 may provide pyrolysis material from the heating chamber 112 to the burner 122.

A collector electrode 126 may be positioned in the FID 120, above the burner 122, where combustion of the pyrolysis material in the flame may affect electrical flow through the collector electrode 126 (e.g., by generation of ions from pyrolysis material combustion). A current measuring circuit 140 may be connected to the collector electrode 126 to measure changes in electrical flow through the collector electrode 126, which may be correlated with the amount and type of combustion material, and thus also correlated with the amount and type of pyrolysis material from pyrolysis of the rock sample 114. For example, the current measuring circuit 140 may send current (or voltage) measurements to a computing system 150, where the computing system 150 may have computer readable software instructions for interpreting the measurements from the current measuring circuit 140 and correlating such measurements with the pyrolysis process (e.g., correlating a measured change in current with an amount of pyrolysis material generated from the rock sample 114 during pyrolysis).

The computing system 150 may be in communication with at least one of the current measuring circuit 140, the FID 120, and the pyrolysis oven 110. From such communication, data relating to the operation of each element may be sent to the computing system 150 for interpretation and correlation, and/or commands for operation of each element may be sent from the computing system 150 to the elements. For example, temperature measurements may be sent from the pyrolysis oven 110 and/or FID 120 to the computing system 150, and current measurements may be sent from the current measuring circuit 140 to the computing system 150, where the computing system 150 may have computer readable instructions for correlating the changes in current measurements with the changes in temperature. As another example, the computing system 150 may have an accelerated heating schedule in the form of executable instructions, which may be sent as commands to the pyrolysis oven 110 to control the oven's temperature and rate of temperature change according to the accelerated heating schedule. In some embodiments, the computing system 150 may also be in communication with gas sources (e.g., the inert gas source 130 and/or the fuel gas source 124) to control the amount of gas that flows into the system 100. In some embodiments, the computing system 150 may also be in communication with additional elements not shown in the system 100, e.g., additional detectors and/or additional pyrolysis ovens.

The elements in the system 100 may be directly and/or indirectly in communication with each other. For example, the FID 120 may be in direct fluid communication with the heating chamber 112 of the pyrolysis oven 110, or pyrolysis material may be flowed through one or more additional measurement devices prior to being flowed to the FID 120. In another example, the computing system 150 may be in direct communication (e.g., wired or wireless) with one or more elements in the system 100, or the computing system 150 may be in indirect communication through one or more transmission nodes with element(s) of the system 100.

In some embodiments, a computing system 150 may be located at a remote location from the pyrolysis oven 110 and FID 120, such that data collected is processed at the remote location. For example, data may be transferred between a computing system 150 and one or more other elements of the system 100 (e.g., the pyrolysis oven 110, the FID 120, and/or the current measuring circuit 140) using a portable memory storage device, such as a jump drive, or wirelessly. In some embodiments, the computing system 150 may be implemented on remote or handheld devices (e.g., laptop computer, smart phone, personal digital assistant, tablet computer, or other mobile device), desktop computers, servers, blades in a server chassis, or any other type of computing device or devices that includes at least the minimum processing power, memory, and input and output device(s) to perform one or more embodiments of the invention.

A computing system 150 may include one or more computer processor(s) 151, non-persistent storage 152 (e.g., random access memory (RAM), cache memory, or flash memory), one or more persistent storage 153 (e.g., a hard disk), a communication interface 154, and numerous other elements and functionalities. The computer processor(s) 151 may be an integrated circuit for processing instructions. The computing system 150 may also include one or more input device(s) 155, such as a touchscreen, keyboard, mouse, microphone, touchpad, electronic pen, or any other type of input device. Further, the computing system 150 may include one or more output device(s) 160, such as a screen (e.g., a liquid crystal display (LCD), a plasma display, or touchscreen), a printer, external storage, or any other output device. One or more of the output device(s) may be the same or different from the input device(s).

The computing system 150 may be connected to a network system (e.g., a local area network (LAN), a wide area network (WAN) such as the Internet, mobile network, or any other type of network) via a network interface connection (not shown). For example, the input device 155 may be coupled to a receiver and a transmitter used for exchanging communication with one or more peripherals connected to the network system. The receiver may receive information relating to one or more rock samples. The transmitter may relay information received by the receiver to other elements in the computing system 150. Further, the computer processor(s) 151 may be configured for performing or aiding in implementing the processes described herein.

Further, one or more elements of the aforementioned computing system 150 may be located at a remote location and be connected to the other elements over the network system. The network system may be a cloud-based interface performing processing at a remote location from the well site and connected to the other elements over a network.

The computing system 150 may implement and/or be connected to a data repository. For example, one type of data repository is a database. A database is a collection of information configured for ease of data retrieval, modification, re-organization, and deletion. In some embodiments, the database includes published/measured data relating to the method and the system as described herein (e.g., oven temperature, changes in temperature, changes in current or voltage readings, pyrolysis material amount correlations, and other rock sample analysis data).

Computer readable software instructions for performing accelerated pyrolysis methods according to embodiments of the present disclosure may be implemented on and/or stored on the computing system 150. The computing system 150 may send commands to one or more elements in the system 100, such as the pyrolysis oven 110 and the FID 120, to perform an accelerated pyrolysis method disclosed herein. For example, the computing system 150 may implement instructions for controlling a temperature in the pyrolysis oven 110 according to an accelerated heating schedule that includes two heating phases (e.g., a first heating phase of holding at an initial temperature and a second heating phase for increasing the temperature at an accelerated rate to a final temperature), and/or instructions for generating a plot of an amount of detected pyrolysis material as a function of time based on changes in measured current from the current measuring circuit 140.

Accelerated pyrolysis methods according to embodiments of the present disclosure may include heating a prepared rock sample in a pyrolysis oven in two heating phases, including first heating phase, where the rock sample may be heated at an initial temperature for a period of time, and a second heating phase, where the temperature of the rock sample may be increased at an accelerated rate (e.g., between 50° C. per minute and 60° C. per minute) until the rock sample reaches a final temperature. Pyrolysis material generated from the rock sample during the heating phases may be flowed to a fluidly connected detector (e.g., FID 120) to detect the amounts of pyrolysis material generated. The amounts of detected pyrolysis material may then be used for analysis of the rock sample (e.g., through analysis of a pyrogram of the detected pyrolysis material).

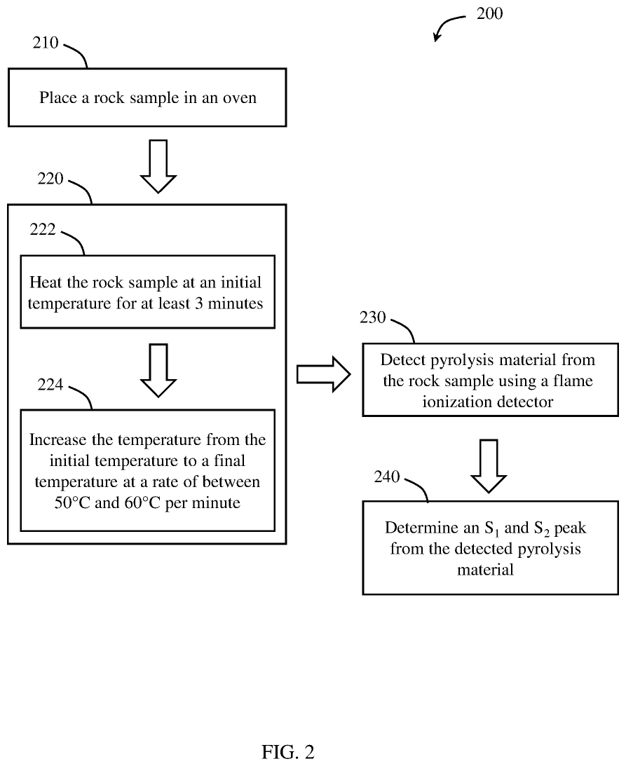

For example, FIG. 2 shows an example of a pyrolysis method according to embodiments of the present disclosure. In some embodiments, one or more of the steps shown in FIG. 2 may be omitted, repeated, and/or substituted.

As shown in FIG. 2, a method 200 may include placing a rock sample in an oven (step 210). Prior to placing in the oven, the rock sample may be prepared, for example, by grinding a rock to a powder. In some embodiments, the rock sample may be ground to a powder having a size less than 100 mesh, e.g., ranging from 30 to 80 mesh, ranging from 40 to 60 mesh, or ranging between 45 and 50 mesh. Depending on the size and type of oven and the type of rock sample, an amount of the rock sample placed in the oven may range, for example, from about 30 to 100 mg (e.g., 40 to 50 mg).

In some embodiments, a portion of the rock sample may be used for a separate carbon assessment in order to determine a percent total organic carbon (TOC) of the rock sample. For example, a rock sample may be ground to a powder, where a portion of the powdered rock sample may be tested to determine the TOC of the rock sample, and another portion of the powered rock sample (e.g., between 30 and 100 mg) may be placed in an oven for pyrolysis. A carbon assessment of the TOC in the rock sample may be performed prior to, during, or after the pyrolysis method 200. In some embodiments, a carbon assessment to determine TOC may include combusting the rock sample in a furnace, where the evolved carbon fractions may be measured and converted to TOC as a mass weight percent of the rock sample.

Rock samples may be taken from different kinds of rocks of interest including, for example, sedimentary rock, coal, and carbonate rocks. Rock samples may also include different types of additives within the rock, such as synthetic polymers and additives used in drilling fluids. For example, rock samples may be taken from a formation of interest in a drilling operation (e.g., by coring) to study the hydrocarbon content in the formation. When the rock sample is taken from an area of the formation already subjected to one or more drilling operations, the collected rock samples may include drilling fluid remnants within the rock sample. In some embodiments, multiple rock samples may be prepared from rocks taken from different locations in a formation of interest to compare hydrocarbon content at the different locations, where each rock sample may undergo separate pyrolysis processes to analyze hydrocarbon content in each location.

A rock sample may be held in an oven under an inert environment. For example, in some embodiments, after the rock sample is positioned in the oven, an inert gas may be pumped into the oven to fill the volume surrounding the rock sample, and any air the inert gas replaced may be pumped out. In some embodiments, a rock sample may be enclosed in a heating chamber in the oven, where all the oxygen within the enclosed heating chamber may be removed (e.g., and replaced with an inert gas) to provide the inert environment around the rock sample. In the inert environment, the rock sample may be heated according to accelerated heating schedules described herein, such as shown in step 220 in FIG. 2.

An accelerated heating schedule 220 according to embodiments of the present disclosure may include heating the rock sample in the oven in a first heating phase at an initial temperature for a first time period, such as shown in step 222. For example, the first heating phase may include holding the oven temperature at the initial temperature, such that the rock sample may be constantly heated at the initial temperature for the first time period. In some embodiments, the initial temperature may be selected from the range of about 300° C. to 350° C. In some embodiments, the first heating phase may include holding the temperature in the oven at the initial temperature for a first time period of at least 3 minutes. For example, a first time period may be selected from a range of 3 to 5 minutes (e.g., 3 minutes, 4 minutes, or 4.5 minutes). In some embodiments, a first heating phase may include heating the rock sample in the oven under an inert environment at an initial temperature of about 300° C. for between about 3 and 4 minutes.

After holding the rock sample in the oven at an initial temperature for a first time period in the first heating phase (step 222), the accelerated heating schedule 220 may include a second heating phase (step 224), in which the temperature in the oven may be increased from the initial temperature to a final temperature at an accelerated rate, such as a rate of greater than 45° C. per minute. For example, a rate of increasing the temperature in the second heating phase may be selected from a range of 50° C. per minute to 60° C. per minute. A final temperature may be selected from a temperature within the range of 650° C. to 800° C., for example, between 650° C. and 750° C., between 700° C. and 800° C., or between 725° C. and 775° C. For example, in some embodiments, a second heating phase may include increasing the temperature from the initial temperature of the first heating phase to a final temperature of about 750° C. at a rate of about 50° C./minute.

During the accelerated heating schedule 220, decomposition of organic matter from the rock sample (pyrolysis material) may be detected by a flame ionization detector (FID), as shown in step 230. Pyrolysis material may be detected, for example, from changes in an electrical current or electrical potential through the FID as pyrolysis material is burned in the FID. In step 240, an S₁ peak and an S₂ peak may be determined from the detected pyrolysis material. For example, the changes in current or voltage detected during pyrolysis may be plotted as a function of time on a pyrogram. A curve may be fitted on the data points, where the resulting curve on the pyrogram may have multiple relative peaks, including an S₁ and S₂ peak. The S₁ and S₂ peaks may be determined as the first occurring relative peak and second occurring relative peak, respectively.

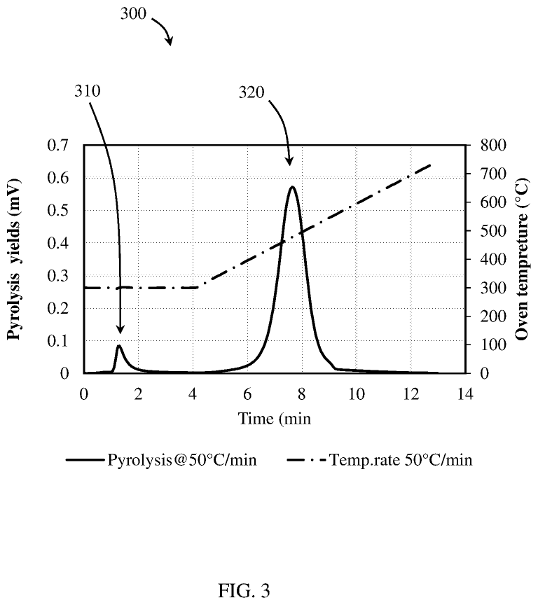

FIG. 3 shows an example of a pyrogram 300 generated from a pyrolysis method according to embodiments of the present disclosure. The pyrogram 300 shows a plot of the amount of pyrolysis material detected during pyrolysis (measured in terms of a change in voltage detected in an associated FID) as a function of time. The curve of the pyrolysis method measurements has a first relative peak in voltage occurring between 1 and 2 minutes of the pyrolysis method, which is the S₁ peak 310. The curve of the pyrolysis method measurements also has a second relative peak in voltage occurring between about 5 and 9 minutes of the pyrolysis method, which is the S₂ peak 320. In the embodiment shown, the entire accelerated heating schedule of the pyrolysis method took less than 12 minutes to generate pyrolysis material showing an S₁ and S₂ peak.

The FID signals may proportionately represent the amount of hydrocarbons detected by the FID that were generated from pyrolysis of the rock sample, and thus, the FID signal may be converted to an amount of hydrocarbons per rock sample. Typically, FID signals may be converted to an amount of hydrocarbons/rock sample using the units milligram of hydrocarbons per gram of rock sample (mg Hc/g rock). An amount of solvent extractable hydrocarbons (e.g., bitumen) in the rock sample may be derived from the S₁ peak 310 on the plot, and an amount of non-solvent extractable hydrocarbons (e.g., kerogen) in the rock sample may be derived from an S₂ peak 320 on the plot. Further, the S₁ peak may correspond to the amount of free hydrocarbons that evolve from the rock sample without cracking kerogen during the first heating phase, and the S₂ peak may correspond to the amount of residual hydrocarbons resulting from cracking of heavy hydrocarbons and from the thermal breakdown of kerogen in the rock sample during the second heating phase. The amount of residual hydrocarbons from the rock sample represented by the S₂ peak may indicate the potential amount of hydrocarbons that may be produced from continued thermal maturation of the source rock.

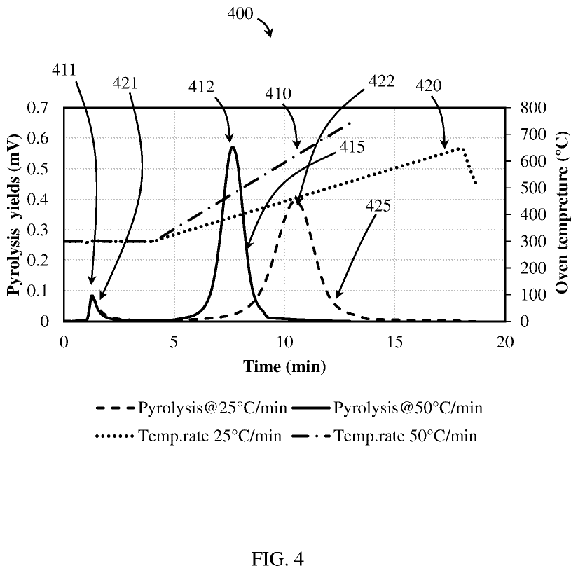

Additionally, a Tₘₐₓ temperature of the rock sample may be determined as the temperature at which the S₂ peak reaches its maximum. In the embodiment shown in FIG. 3, the Tₘₐₓ would be equal to the temperature of the oven when the S₂ peak reached its maximum or apex, shown at just under 6 minutes. According to embodiments of the present disclosure, the temperature of the oven may also be plotted in a pyrogram, such as shown in FIG. 4 (described below), or the temperature of the oven may be recorded and stored as being associated with the detected pyrolysis yields and/or time, which may then be recalled and used in analysis of the rock sample. Tₘₐₓ may be used, for example, to characterize the thermal evolution of the organic matter in the rock sample.

By using accelerated pyrolysis methods and systems according to embodiments of the present disclosure, Tₘₐₓ may be more accurately determined and the rock-eval parameters S₁, S₂, and Tₘₐₓ may be more quickly determined when compared with conventional pyrolysis techniques. For example, FIG. 4 shows a pyrogram 400 comparing a heating schedule 410 and pyrolysis yield 415 from a pyrolysis method according to embodiments of the present disclosure and a heating schedule 420 and pyrolysis yield 425 from a conventional pyrolysis method for powdered rock samples prepared from the same rock.

The accelerated heating schedule 410 according to embodiments of the present disclosure may include subjecting the rock sample to a pyrolysis method lasting less than 15 minutes (e.g., less than 13 minutes, such as for the heating schedule shown in FIG. 4). The accelerated heating schedule 410 used in FIG. 4 included a first heating phase of heating the rock sample at an initial temperature of about 300° C. for a first period of time between 3 and 5 minutes (e.g., 4 minutes, as shown in FIG. 4), and a second heating phase of increasing the temperature from the initial temperature to a final temperature of 650° C. at a rate of 50° C. per minute. The pyrolysis yield 415 detected during the first and second heating phases was plotted on the pyrogram 400 and includes an S₁ peak 411 detected at about 2 minutes and an S₂ peak 412 detected between about 5 and 9 minutes. Further, from the S₂ peak, the Tₘₐₓ was determined to be about 428° C. (the temperature of the oven at the apex of the S₂ peak).

The heating schedule 420 of the conventional pyrolysis method included a first heating phase of heating the rock sample at 300° C. for about 8 minutes, and a second heating phase of increasing the temperature at a rate of 25° C. until reaching a final temperature of 650° C. The pyrolysis yield 425 detected during the conventional heating schedule 420 includes an S₁ peak 421 detected at about 5 minutes and an S₂ peak 422 detected between 8 and 14 minutes. Further, from the S₂ peak, the Tₘₐₓ was determined to be about 429° C. (the temperature of the oven at the apex of the S₂ peak).

By using the accelerated heating schedule according to embodiments disclosed herein, the rock-eval parameters S₁, S₂, and Tₘₐₓ may be more quickly determined when compared with conventional pyrolysis methods, which may allow faster analysis of hydrocarbon contents within the rock sample. For example, an amount of solvent extractable hydrocarbons and non-solvent extractable hydrocarbons in the rock sample may be more quickly determined from an S₁ peak and S₂ peak on the plot, respectively, and Tₘₐₓ may be more quickly determined from the S₂ peak on the plot.

According to embodiments of the present disclosure, systems may be provided to automatically run accelerated heating schedules and/or collect and analyze pyrolysis data from an accelerated heating schedule (e.g., to determine rock-eval parameters S₁, S₂, and Tₘₐₓ). For example, a pyrolysis system may include a pyrolysis oven, a FID, and a computing system in communication with the pyrolysis oven and the FID. The computing system may include instructions for controlling a temperature in the pyrolysis oven according to an accelerated heating schedule according to embodiments of the present disclosure. For example, the computer readable instructions may include instructions to initiate a first heating phase, including heating the pyrolysis oven at an initial temperature for between 3 and 4 minutes, and upon completion of the first heating phase, instruction to initiate a second heating phase, including increasing the temperature in the pyrolysis oven at a rate ranging between 50° C. per minute and 60° C. per minute to a final temperature. The computing system may also include instructions for receiving and processing data from the FID. For example, computer readable instructions may include instructions to generate a plot of an amount of detected pyrolysis material from the FID as a function of time. Computer readable instructions may also include instructions to determine an S₁ peak and an S₂ peak on the plot, correlate the temperature in the pyrolysis oven with the time of the heating schedule, and determine Tₘₐₓ from the S₂ peak.

Multiple pyrolysis methods according to embodiments of the present disclosure may be performed simultaneously or sequentially on rock samples prepared from the same rock or on rock samples prepared from different rock samples. By performing multiple pyrolysis methods on rock samples prepared from the same rock, more accurate and comprehensive analysis may be performed on the rock. Further, by using pyrolysis methods according to embodiments of the present disclosure, more rock samples may be tested in a shorter amount of time compared with conventional methods.

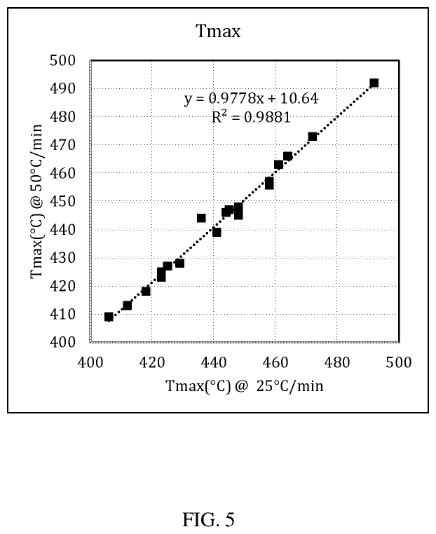

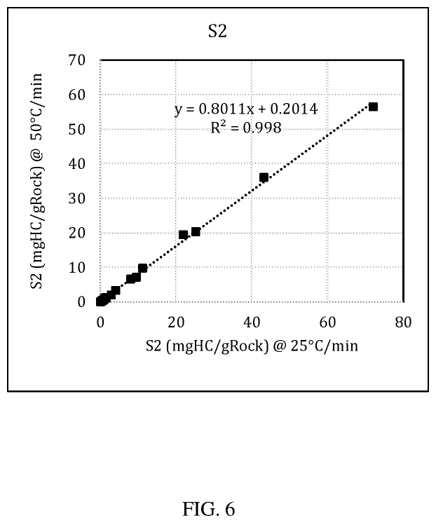

Advantageously, pyrolysis methods according to embodiments of the present disclosure may be performed using conventional pyrolysis equipment, such as used in conventional rock-eval methods, but may provide relatively faster results without sacrificing accuracy. For example, FIGS. 5 and 6 show comparisons of rock-eval parameters determined using pyrolysis methods according to embodiments of the present disclosure and conventional pyrolysis methods. The comparisons show excellent agreement between the results of both methods, thus showing that accelerated heating schedules according to embodiments of the present disclosure may provide substantially the same results as a conventional method, but more quickly.

In FIGS. 5 and 6, 19 samples were each prepared from a rock that was ground to about 50 mesh. About 45 mg of the powdered rock was allocated for each sample for use in a conventional method and a method according to embodiments of the present disclosure for comparison of rock-eval parameters determined from each method. In both pyrolysis methods, the samples were heated in a first heating phase at 300° C. prior to being heated in a second heating phase. The samples analyzed using a conventional method were heated in a second heating phase using a conventional heating rate of 25° C./minute, while comparison samples were heated in a second heating phase using an accelerated heating rate of 50° C./minute according to embodiments of the present disclosure.

FIG. 5 shows the measured Tₘₐₓ values determined for each of the samples using the two methods. As shown, there was a near linear correlation (with minimum deviation) between the results, showing good agreement between the derived Tₘₐₓ values using both methods. Thus, FIG. 5 shows that methods of the present disclosure using an accelerated heating rate may more quickly determine Tₘₐₓ values without sacrificing accuracy.

FIG. 6 shows a correlation between S₂ values generated using the conventional heating rate of 25° C./minute and S₂ values generated using the heating rate of 50° C./minute according to methods disclosed herein. As shown, there was a near linear correlation (with minimum deviation) between the results, showing good agreement between the S₂ values using both methods. Thus, FIG. 6 shows that methods of the present disclosure using an accelerated heating rate may more quickly determine S₂ values without sacrificing accuracy.

By using pyrolysis methods according to embodiments of the present disclosure, rock may be characterized more quickly without sacrificing accuracy. By more quickly characterizing rock samples, well development and planning may be expedited, thereby reducing costs in hydrocarbon extraction operations. Further, methods of the present disclosure may provide more pronounced S₂ peaks, which may improve determination of the Tₘₐₓ value in source rock samples having high thermal maturity or bad preservation during diagenesis. In contrast, determining the Tₘₐₓ value for samples with very low reactive kerogen, for example, in overmature source rock has been challenging in conventional pyrolysis methods, as the S₂ peaks generated from conventional pyrolysis methods may be flattened or plateaued.

Further, pyrolysis methods according to embodiments of the present disclosure may be performed using already existing equipment. Thus, rock characterization may be more quickly performed using accelerated pyrolysis methods disclosed herein without incurring additional equipment costs.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method, comprising:
placing a rock sample in an inert environment in an oven;
heating the rock sample in the oven at about 300° C. for between about 3 and 4 minutes;
increasing the temperature to about 750° C. at a rate ranging between 50° C. per minute and 60° C. per minute; and
detecting pyrolysis material from the rock sample by a flame ionization detector.

2. The method of claim 1, wherein prior to placing the rock sample in the oven, the rock sample is ground to a powder having a size ranging from 40 to 60 mesh.

3. The method of claim 1, wherein an amount of the rock sample placed in the oven ranges from about 40 to 100 mg.

4. The method of claim 1, wherein the rock sample comprises a sedimentary rock and at least one of a synthetic polymer and a drilling fluid additive.

5. The method of claim 1, further comprising generating a pyrogram comprising a plot of an amount of the detected pyrolysis material as a function of time.

6. The method of claim 5, further comprising:
deriving an amount of solvent extractable hydrocarbons in the rock sample from an S₁ peak on the plot;
deriving an amount of non-solvent extractable hydrocarbons in the rock sample from an S₂ peak on the plot; and
determining a Tₘₐₓ temperature of the sample as the temperature at which the S₂ peak reaches its maximum.

7. The method of claim 1, further comprising determining a percent total organic carbon of the rock sample.

8. A method, comprising:
subjecting a rock sample to a pyrolysis process lasting less than 15 minutes, the pyrolysis process comprising:
a first heating phase comprising heating the rock sample at an initial temperature of about 300° C.;
a second heating phase comprising heating the rock sample from the initial temperature to a final temperature ranging between 650° C. and 750° C. at a rate of 50° C. per minute; and
detecting pyrolysis material from the rock sample during the first and second heating phases;
generating a pyrogram comprising a plot of an amount of the detected pyrolysis material as a function of time.

9. The method of claim 8, wherein the rock sample is heated in an pyrolysis oven comprising a heating chamber and a flame ionization detector fluidly attached to the heating chamber.

10. The method of claim 8, wherein in the first heating phase, the rock sample is heated at the initial temperature for about 3 minutes.

11. The method of claim 8, further comprising:
deriving an amount of solvent extractable hydrocarbons in the rock sample from an S₁ peak on the plot;
deriving an amount of non-solvent extractable hydrocarbons in the rock sample from an S₂ peak on the plot; and
determining a Tₘₐₓ temperature of the rock sample as the temperature at which the S₂ peak reaches its maximum.

12. The method of claim 8, wherein the rock sample is heated in the pyrolysis process under inert conditions.

13. The method of claim 8, further comprising:
grinding a rock to between 40 and 60 mesh to make the rock sample; and
subjecting between 40 and 100 mg of the rock sample to the pyrolysis process.

14. The method of claim 8, wherein the rock sample is selected from a sedimentary rock and coal.

15. A system, comprising:
a pyrolysis oven;
a flame ionization detector, comprising:
a burner fluidly connected to a heating chamber in the pyrolysis oven and a fuel gas; and
a collector electrode positioned above the burner; and
a computing system in communication with the pyrolysis oven and the flame ionization detector, the computing system comprising:
instructions for controlling a temperature in the pyrolysis oven according to a heating schedule, comprising:
heating the pyrolysis oven at an initial temperature for between 3 and 4 minutes in a first heating phase; and
in a second heating phase, increasing the temperature in the pyrolysis oven at a rate ranging between 50° C. per minute and 60° C. per minute to a final temperature;
instructions for generating a plot of an amount of detected pyrolysis material as a function of time based on changes in measured electrical flow through the collector electrode.

16. The system of claim 15, wherein the initial temperature ranges from about 300° C. to 350° C.

17. The system of claim 15, wherein the final temperature ranges from about 650° C. to 750° C.

18. The system of claim 15, wherein the computing system further includes instructions for determining an S₁ peak and an S₂ peak on the plot.

19. The system of claim 18, wherein the computing system further includes instructions for correlating the temperature in the pyrolysis oven with the time.

20. The system of claim 19, wherein the computing system further includes instructions for determining a Tₘₐₓ temperature of the rock sample as the temperature at which the S₂ peak reaches its maximum.
